(19) United States
(12) Patent Application Publication
(10) Pub. No.: US 2013/0177542 A1
(43) Pub. Date: Jul. 11, 2013

(54) Osteoporosis Treatment Means and Method

(71) Applicant: Complete Body Health, LLC, Rancho Palos Verdes, CA (US)

(72) Inventors: Barbara Gene Veit Hartl, Rancho Palos Verdes, CA (US); Clairice Gene Tipton Veit, Encino, CA (US)

(73) Assignee: Complete Body Health, LLC, Rancho Palos Verdes, CA (US)

(21) Appl. No.: 13/679,180

(22) Filed: Nov. 16, 2012

Related U.S. Application Data
(60) Provisional application No. 61/561,150, filed on Nov. 17, 2011.

(51) Int. Cl. A61K 9/00

(52) U.S. Cl. CPC A61K 9/0087
USPC 424/94.1; 424/601; 514/690; 424/643

(57) ABSTRACT

Supplements (non-hormonal) known to aid in bone formation and a method of spreading the supplements out throughout the day taking into consideration counteracting effects some have on others and according to when the body is more likely to need them while keeping in mind consumer compliance, which consensus suggests 3 times per day would be a maximum. (including in some instances an additional bed-time capsule).

### BACKGROUND OF THE INVENTION

### Field of Invention

This invention relates to supplements to help the body maintain good body health and particularly to reducing or preventing Osteoporosis

### SUMMARY OF THE INVENTION

The objective is to provide a product that provides all the elements (supplements—vitamin, minerals . . . ) that aid in bone formation and hence retard or reverse osteoporosis.

### DETAILED DESCRIPTION OF THE INVENTION

Bone is under a constant process of resorption (breakdown), and formation. Our bodies do this as a natural way to keep our bones young (new), strong, and healthy. After a peak bone mass is achieved, bone mass remains stable (resorption and formation are equal). As we age, bone formation slows and the balance shifts between how fast we break down the bone (resorption), and how fast we build bone. When your body does not build bone as fast as it breaks it down, it leads to bone loss, weakness and osteoporosis.

Since bone formation is a constant process, it is ideal if all of the substances known to aid in the process are present at all times.

The challenges in developing a system for treating osteoporosis or bone loss are the following:
1) It must address the difficulty of the body's ability to absorb vitamins and minerals,
2) It must consider the synergistic and opposing effects of the different substances
3) There is the need for constant availability of the substances
4) Maximize consumer compliance given that many supplements are water soluble and hence, excreted rapidly from the body

This system takes all supplements (non-hormonal) known to aid in bone formation and spreads them out throughout the day taking into consideration counteracting effects some have on others and according to when the body is more likely to need them (for example B vitamins, which are energy enhancers, are consumed in the earlier part of the day) while keeping in mind consumer compliance, which consensus suggests 3 times per day would be a maximum. (including in some instances an additional bed-time capsule).

Since this is designed to replace the multiple vitamins and minerals the bone specific supplements may be combined and included in a capsule/capsules that have some of the more general health supplements included. Additionally some of the supplements (such as vitamin B as one example) are in both categories (general health and necessary for bone formation) but are not in the same capsule with the calcium and other minerals necessary for bone formation. Some supplements are not specific to bone formation, but are part of the multiple vitamin aspect of this system.

All of the Vitamin B's, biotin and folic acid are taken in the earlier part of the day because of the energy producing quality of these supplements.

Vitamin C is taken throughout the day. There are water soluble and fat soluble forms. Both will be part of this protocol. The fat soluble will stay in the body longer (this is ascorbyl palmitate) and the water soluble form will be a buffered form of Vitamin C. The vitamin C may/will be coupled into a capsule with other supplements

Calcium and other minerals (except strontium) will be taken three times per day. Research has shown that calcium absorption always goes up with the addition of other minerals. Since most bone formation occurs at night, Calcium will be given at dinner and bedtime. It will also be given at breakfast time. Since strontium interferes with calcium absorption it will be taken at a different time of day lunch. Strontium aids in bone retention. The mechanisms are less clear than for some of the other elements, however, it should be part of a comprehensive bone formula. For this reason, I have excluded Calcium at lunch, replaced it with strontium and put Calcium as a FOURTH interval for taking supplements—bedtime.

Chondroitin, glucosamine and MSM are added for joints, while not necessary for bones. If the # of capsules becomes too large, these will be omitted.

The amino acids, Acetyl carnitine and taurine are not necessarily bone specific and are added here because they are beneficial to overall health. Lipoic acid is coupled with the amino acids, but may be encapsulated with any other supplement or group of supplements as appropriate. When these are taken is variable, but should be taken throughout day.

Strontium is very likely to be packaged as it's own capsule and will be taken only one time per day because of its interference with Calcium. In order to maximize consumer compliance, it will not be taken more than the one time per day.

Fat Soluble Vitamins:

since they embed into cell membranes, they are stored and not secreted. But also because they are fat soluble, they need to be taken with meals or with other fats. These will be combined to the extent that packaging allows and that there are no conflicts. For example: Vitamin A should not be taken with vitamin D so they would not be packaged together. The time of day these are taken may be modified as it becomes clear how they will be packaged.

Vitamin E is divided into tocotrienols and tocopherols. The better of the two is believed to be the tocotrienols and they should be taken at night. Hence the tocotrienols are taken with dinner, the tocopherols are taken in the morning. Mixed carotenoids may be able to be packaged with other supplements since they are not a gel cap or physical oil. If this is the case they may be taken throughout the day rather than at one time. However, because they are fat soluble, they can be consumed all at once.

CoQ10 or CoQH are oils and very possible need to have their own gel cap. CoQH is the preferred form, however, it may be too costly.

Vit K. either MK-7 or MK-4 as Vitamin K2 can be used. I may also use vitamin K1 as well. If these can be combined with other fat soluble vitamins, it will be done.

Omega oils will be combined and may be combined with any of the fat soluble vitamins if permitted.

The goal is to make this a user compliant as possible while keeping the steady input of vitamins and minerals into the body in their most absorbable, bioavailable forms.

Many ingredients will be combined together to yield as few different capsules/tables/capliques as possible (determined by reactivity, solubility, compatibility, dosage, time of day to take).

This system will likely be a “kit” with several containers with the various grouped supplements. The “lunch’ pills, may come grouped in a packet so the consumer an just grab the whole thing and be “on the go . . . ”

The Water soluble Supplements, Amino acids, fat soluble supplements and oils together with the ranges thereof and when they should be taken are shown in the Listing 1 and Notes 1 attached. Also The Water soluble Supplements, Amino acids, fat soluble supplements and oils together with the preferred amounts thereof and when they should be taken are shown in the Listing 2 and Notes 2 below.

The quantities of the supplements are given on the Listings 1 and 2 below and depend upon which sources or chelates are used.

Optimally consumption will not be more than 4 times per day with the exception of strontium which at the consumer's discretion may choose when to take it according to the guidelines that it. not be taken within a 4 hour proximity to calcium.

Depending upon formulation, ideally tablets or capsules will be made and broken down as follows:

Acetyl-1

carnitine

B2

B3

B5

B6

B12

Biotin

Folic Acid

Boron

In one capsule that can be taken at breakfast and lunch with the indicated daily dosage distributed.

Vitamin C

Calcium

Chromium

Chondroitin

Glucosamine

Iodine

lipoic acid

manganese

magnesium

Molybdenum

MSM

Phosphorus

Potassium

selenium

silicon

taurine

zinc

Alanine

Arginine

Aspartic acid

Cystine/Cysteine

Glutamic acid

Glycine

Histidine

Isoleucine

Leucine

Lysine

Methionine

Phenylalanine

Proline

Serine

Threonine

Tryptophan

Tyrosine

Valine

Choline

PABA

Hesperidin

Trace minerals on naturally occurring ratios in sea salt The above would also be made into capsule form with evenly distributed daily dosage (it will be probable that there will be several capsules required at each recommended time of consumption). The dosage would be distributed into 4 capsules that would be taken according to the spreadsheet (breakfast, lunch, dinner and bed time)

Vitamin A

Vitamin E as tocop

Mixed carotenoids

COQ10

Vit K

omega 3

Omega 6

These oils, depending upon formulation requirements, will either be packaged all together or separated into different gel caps. Since they are fat soluble, they can be consumed only once per day, at present time the recommendation is in the morning

Vit D3 This is also fat soluble so the entire dose can be taken at one time. It is recommended that it be a different time from the above which includes vitamin A and so the recommendation I to take it in the evening.

Vitamin E: tocotrienols: to be taken at bed time

LISTING 1

| H20 | | | | | | | |
|---|---|---|---|---|---|---|---|
| Soll | Supplement | Amount | | breakfast | lunch | dinner | bed |
| | B! | 100-300 | mg | x | x | | |
| | B2 | 50-300 | mg | x | x | | |
| | B3 | 10-200 | mg | x | x | | |
| | B5 | 50-300 | mg | x | x | | |
| | B6 | 75-200 | mg | x | x | | |
| | B12 | 800 mcg-1.5 mg | | x | x | | |
| | Biotin | 1-5 | mg | x | x | | |
| | Folic Acid | 200 mcg-1.0 mg | | x | x | | |
| | PABA | 15-100 | mg | x | x | | |
| | Vitamin C | 3000-6000 | mg | | | | |
| | Vitamin C | H20 | | x | x | | |
| | Vitamin C | as.pa | | x | x | x | |
| | Calcium | 800-2000 | mg | x | x | x | |
| | (elemental) | | | | | | |
| | magnesium | 300-1000 | mg | x | x | x | |
| | Molybdenum | 350-550 | mcg | x | x | x | |
| | Phosphorus | 400-1000 | mg | x | x | x | |
| | Potassium | 150-400 | mg | x | x | x | |
| | selenium | 200-300 | mcg | x | x | x | |
| V | zinc | 20-80 | mg | x | x | x | x |
| | manganese | 2-8 | mg | x | x | x | |
| | Chromium | 100-400 | mcg | x | x | x | |
| V | Boron | 500 mcg-20 mg | | x | x | x | |
| | silica | 200-500 | mg | x | x | x | |
| | silicon | 5-20 | mg | x | x | x | |
| | manganese | 2-8 | mg | x | x | x | |
| | 70+ trace | naturally | | x | x | x | |
| | minerals | occur. | | | | | |
| | copper | 2.5 | mg | x | x | x | |
| | Iodine | 1-50 | mg | x | x | x | |
| | lipoic acid | 200-500 | mg | x | x | x | |
| | Chondroitin | 500-1800 | mg | x | x | x | |
| | Glucosamine | 1-2 | g | x | x | x | |
| | MSM | 800-1200 | mg | | | | |
| amino | taurine | 500-3000 | mg | x | x | x | |
| acids+ | Acetyl-l | 500-1500 | mg | x | x | x | |
| | carnitine | | | | | | |
| | Alanine | 150-200 | mg | x | x | x | |
| | Arginine | 220-300 | mg | x | x | x | |
| | Aspartic acid | 250-300 | mg | x | x | x | |
| | Cystine/ | 50-70 | mg | x | x | x | |
| | Cysteine | | | | | | |
| | Glutamine | up to 15 | g | x | x | x | |
| | Glutamic acid | 400-550 | mg | x | x | x | |
| | Glycine | 100-200 | mg | x | x | x | |
| | Histidine | 40-80 | mg | x | x | x | |
| | Isoleucine | 100-200 | mg | x | x | x | |
| | Leucine | 200-300 | mg | x | x | x | |
| | Lysine | 75-175 | mg | x | x | x | |
| | Methionine | 70-125 | mg | x | x | x | |
| | Phenylalanine | 120-190 | mg | x | x | x | |
| | Proline | 120-185 | mg | x | x | x | |
| | Serine | 130-190 | mg | x | x | x | |
| | Threonine | 75-150 | mg | x | x | x | |
| | Tryptophan | 30-60 | mg | x | x | x | |
| | Tyrosine | 100-200 | mg | x | x | x | |
| | Valine | 100-200 | mg | x | x | x | |
| | Choline | 75-1150 | mg | x | x | x | |
| | strontium | 0-680 | mg | | | | x |
| Fat | Vit E | 50-300 | mg | | | x | |
| sol | tocotrienols | | | | | | |
| | Vit D3 | 3000-6000 | IU | | | x | |
| | Vitamin A | 2,000-5000 | iU | x | | | |
| | Vit E | 100-600 | IU | x | | | |
| | tocopherols | | | | | | |
| | mixed | <30,000 | IU | x | | | |
| | carotenoids | | | | | | |
| | CoQ10 or CoQH | 100-500 | mg | x | | | |

| | | | | |
|---|---|---|---|---|
| | Vit K | 150 mcg-60 mg | x | (x) |
| oils | omega 3 | 1 g-2 g | x | |
| | | tsp-2 tsp | | |
| | Omega 6 | 3 g-5 g | x | |

Notes 1 to Listing 1

between buffered H2O soluble and fat soluble up to 6000 mg

between buffered H2O soluble and fat soluble (ascorbyl palmitate) up to 6000 mg

calcium as calcium citrate/malate, as calcium carbonate, calcium bis-glycinate, MCHC (macrocrystalline hydroxy appetite) or any other source of calcium may be used to meet this elemental amount. Absorption of tablet form of calcium is an issue—powder or capsule is preferred. (2000 mg is tolerable upper) as 1-selenomethionine, Selenium seLECT or as L-Se-methylselenocysteine) or any other chelated form chelated zinc may be in the form of chromium picolinate or any other form

upper limit should be 20 mg as magnesium silicate, but prefer to use silicon as mentioned

preferred sources are ch-OSA (choline stabilized orthosalicic acid-Biosil) or Jarrowsil: Activated Silicon formula of molecular clusters of stabilized silicic acid. These are the preferred sources however they are both patented products and depending upon user agreements may not be the sources available for use here.

naturally occurring jpercentages of trace minerals from sea salt 2.5 mg

Both Lugol's solution and Ioderal are one-third molecular iodine (5%) and two-thirds potassium iodide (10%). Studies done to date indicate that the best iodine supplement is one that includes molecular iodine (I2), which breast tissue prefers. this may be of mixture of r-lipoic and alpha lipoic or one or the other Chondroitin, glucosamine and MSM are all taken for joints, Depending upon the size of the supplements etc, these three may be omitted from the formula if logistically difficult or if costs become too great see notes under chondroitin

typical doses are for 1500 mg/day, however, this is not a bone-specific supplement and the amount may be reduced for packaging or cost purposes strontium interferes with calcium absorption (but helps with bone retention). Strontium needs to be taken separately from calcium; therefore calcium is omitted at this interval to accommodate strontium and the amount of strontium will likely be reduced as a result of being taken only once and in view that the goal is not to have the consumer take the supplements more than 4 times per day. (3 would be ideal but virtually impossible).

Strontium will likely be around 300 mg. annatto is the preferred source because has highest delta tocotrienol amount: it is 90% delta, 10% gamma (the smallest of the tocotrienols). Also preferred because it contains no tocopherol. Vit Es are fat soluble so take with meals not with tocopherols because alpha tocopherol will compete with the tocotrienols for binding—also these are very good for reducing cholesterol, cholecalciferol fat soluble so taken with meals, not with vit A because of interference in absorption as retinyl palmitate are fat soluble so take with meals. Do not take with the tocotrienols because they will Interfere with the tocotrienols' ability to bind preferred source is from Dunaliella Salina algae ubiquinol (CoQH)is reduced form of COQ10 and is preferred for older people because the ability to reduce COQ10 decreases with increasing age. Since osteoporosis will affect mostly older people, ubiquinol is preferred (however it is about 3× the price and may be cost prohibitive) dosage for K2 as MK-7 will be between 150-250 mcg. If using MK-4 the dosage is between 40-60 mg. I may also add K1

if Omega 3 is krill oil (preferred) Krill oil is preferred because smaller quantity yields more omega 3, and also more pure with no mercury, and does not have the fishy taste. However cost may be a factor if Omega 3 is cod liver oil primrose oil preferred over borage oil

The ranges above are give or take 20%, however ratios between some of the elements can be important and also certain items should not be lowered too much or the user will not get the bone growth that is desired. As for the oils, 2 capsules would be minimum with 8 being maximum.

LISTING 2

| H20 | | | | | | | |
|---|---|---|---|---|---|---|---|
| Soll | Supplement | Amount | | breakfast | lunch | dinner | bed |
| | B! | 100 | mg | x | x | | |
| | B2 | 200 | mg | x | x | | |
| | B3 | 100 | mg | x | x | | |
| | B5 | 200 | mg | x | x | | |
| | B6 | 100 | mg | x | x | | |
| | B12 | 1.2 | mg | x | x | | |
| | Biotin | 4 | mg | x | x | | |
| | Folic Acid | 600 | mcg | x | x | | |
| | PABA | 80 | mg | x | x | | |
| | Vitamin C | | | | | | |
| | Vitamin C | H20 2000 | mg | x | x | | |
| | Vitamin C | as.pa 300 | mg | x | x | x | |
| | Calcium | 1500 | mg | x | x | x | |
| | (elemental) | (elemental) | | | | | |
| | magnesium | 1000 | mg | x | x | x | |
| | Molybdenum | 500 | mcg | x | x | x | |
| | Phosphorus | 800 | mg | x | x | x | |
| | Potassium | 150 | mg | x | x | x | |
| | selenium | 225 | mcg | x | x | x | |
| | zinc | 25 | mg | x | x | x | x |
| | manganese | 5 | mg | x | x | x | |
| | Chromium | 200 | mcg | x | x | x | |
| | Boron | 3 | mg | x | x | x | |
| | silica | 200-500 | mg | x | x | x | |
| | silicon | 5-20 | mg | x | x | x | |
| | manganese | 5 | mg | x | x | x | |
| | 70+ trace | naturally | | x | x | x | |
| | minerals | occur. | | | | | |
| | copper | 3 | mg | x | x | x | |
| | Iodine | 20 | mg | x | x | x | |
| | lipoic acid | 300 | mg | x | x | x | |
| | Chondroitin | 1200 | mg | x | x | x | |
| | Glucosamine | 1500 | mg | x | x | x | |
| | MSM | 1000 | mg | | | | |
| amino | taurine | 500 | mg | x | x | x | |
| acids+ | Acetyl-l | 500 | mg | x | x | x | |
| | carnitine | | | | | | |
| | Alanine | 181 | mg | x | x | x | |
| | Arginine | 257 | mg | x | x | x | |
| | Aspartic acid | 274 | mg | x | x | x | |
| | Cystine/ | 61 | mg | x | x | x | |
| | Cysteine | | | | | | |
| | Glutamine | up to 15 | g | x | x | x | |
| | Glutamic acid | 449 | mg | x | x | x | |
| | Glycine | 147 | mg | x | x | x | |
| | Histidine | 69 | mg | x | x | x | |
| | Isoleucine | 126 | mg | x | x | x | |
| | Leucine | 257 | mg | x | x | x | |
| | Lysine | 122 | mg | x | x | x | |
| | Methionine | 92 | mg | x | x | x | |
| | Phenylalanine | 156 | mg | x | x | x | |
| | Proline | 157 | mg | x | x | x | |
| | Serine | 161 | mg | x | x | x | |
| | Threonine | 112 | mg | x | x | x | |
| | Tryptophan | 42 | mg | x | x | x | |
| | Tyrosine | 148 | mg | x | x | x | |
| | Valine | 146 | mg | x | x | x | |
| | Choline | 100 | mg | x | x | x | |
| | strontium | 0-680 | mg | | | | x |
| Fat | Vit E | 200 | mg | | | x | |
| sol | tocotrienols | | | | | | |
| | Vit D 3 | 5000 | IU | | | x | |
| | Vitamin A | 2500 | iU | x | | | |
| | Vit E | 400 | IU | x | | | |
| | tocopherols | | | | | | |
| | mixed | 25,000 | IU | x | | | |
| | carotenoids | | | | | | |
| | CoQ10 or CoQH | 300 | mg | x | | | |

| | | | | |
|---|---|---|---|---|
| | Vit K | 150 mcg-60 mg | x | (x) |
| oils | omega 3 | 1 g-2 g | x | |
| | Omega 6 | 3 g-8 g | x | |

Notes 2 to Listing 2

benlotiamine P5P methylcobalamin upper limit is 1000 mcg http://lpi.oregonstate.edu/infocenter/vitamins/fa/ 2000 buffered C ascorbyl palmitate calcium as calcium citrate/malate, as calcium carbonate, calcium bis-glycinate, MCHC (microcrystalline hydroxy appetite) or any other source of calcium may be used to meet this elemental amount. Absorption of tablet form of calcium is an issue - powder or capsule is preferred, close to 1:1 ratio with Calcium; Mag molybdenum glycinate chelate 700=RDA, 3000 is upper limit—if use MCHA for calcium, there is phosphorus 595mg postassium glconate yields 99mg potassium; use potassium silica complex or postassium glycinate amino acid complex as 1-selenomethionine, Selenium seLECT or as L-Se-methylselenocysteine) (SeMSC) chelated zinc: zinc picolinate or zinc gluconate may be in the form of chromium picolinate or any other form upper limit should be 20mg—as amino acid chelate as magnesium silicate, but prefer to use silicon as mentioned—http:/www.liquiavitaminsleader.com/silica/ preferred sources are ch-OSA (choline stabilized orthosalicic acid-Biosil) or Jarrowsil: Activated Silicon formula of molecular clusters of stabilized silicic acid. These are the preferred sources however they are both patented products and depending upon user agreements may not be the sources available for use here, naturally occurring jpercentages of trace minerals from sea salt 2.5 mg

Both Lugol's solution and Ioderal are one-third molecular iodine (5%) and two-thirds potassium iodide (10%). Studies done to date indicate that the best iodine supplement is one that includes molecular iodine (12), which breast tissue prefers.

this may be of mixture of r-lipoic and alpha lipoic or one or the other Chondroitin, glucosamine and MSM are all taken for joints, Depending upon the size of the supplements etc, these three may be omitted from the formula if logistically difficult or if costs become too great see notes under chondroitin

typical doses are for 1500 mg/day, however, this is not a bone-specific supplement and the amount may be reduced for packaging or cost purposes strontium interferes with calcium absorption (but helps with bone retention). Strontium needs to be taken separately from calcium; therefore calcium is omitted at this interval to accommodate strontium and the amount of strontium will likely be reduced as a result of being taken only once and in view that the goal is not to have the consumer take the supplements more than 4 times per day. (3 would be ideal but virtually impossible). Strontium will likely be around 300 mg. annatto is the preferred source because has highest delta tocotrienol amount: it is 90% delta, 10% gamma (the smallest of the tocotrienols). Also preferred because it contains no tocopherol. Vit Es are fat soluble so take with meals not with tocopherols because alpha tocopherol will compete with the tocotrienols for binding - also these are very good for reducing cholesterol, cholecalciferol fat soluble so taken with meals, not with vit A because of interference in absorption as retinyl palmitate are fat soluble so take with meals. Do not take with the tocotrienols because they will interfere with the tocotrienols' ability to bind preferred source is from Dunaliella Salina algae ubiquinol (CoQH) is reduced form of COQ10 and is preferred for older people because the ability to reduce COQ1Q decreases with increasing age. Since osteoporosis will affect mostly older people, ubiquinol is preferred (however it is about 3× the price and may be cost prohibitive) The addition of d-Limonene to this product CoQHnaturally improves its solubility, dosage for K2 as MK-7 will be between 150-250mcg. If using MK-4 the dosage is between 40-60 mg.

if Omega 3 is krill oil (preferred) Krill oil is preferred because smaller quantity yields more omega 3, and also more pure with no mercury, and does not have the fishy taste. However cost may be a factor if Omega 3 is cod liver oil primrose oil preferred over borage oil

It should be apparent tol in the art that the above embodiment is merely a description of one embodiment of the present invention and that numerous and other varied embodiments could be made without departing from the spirit And scope of the invention.

I claim:

1. A supplement for reducing or preventing osteoporosis comprising taking the constituents of Listing 1.

2. A method for reducing or preventing osteoporosis according to claim 1 comprising taking the constituents of said Listing 1 at the times set forth in said Listing 1.

3. A supplement for reducing or preventing osteoporosis comprising taking the constituents of Listing 2.

4. A method for reducing or preventing osteoporosis according to claim 3 comprising taking the constituents of said Listing 2 at the times set forth in said Listing 2.
